(19) United States
(12) Patent Application Publication
(10) Pub. No.: US 2015/0080598 A1
(43) Pub. Date: Mar. 19, 2015

(54) ASYMMETRIC SYNTHESIS FOR PREPARING FLUOROLEUCINE ALKYL ESTERS

(71) Applicant: Merck Sharp & Dohme Corp., Rahway, NJ (US)

(72) Inventors: Guy Humphrey, Hillsborough, NJ (US); Cheol Chung, Westfield, NJ (US); Nelo Rivera, Milford, NJ (US); Kevin Belyk, Metuchen, NJ (US)

(21) Appl. No.: 14/387,897

(22) PCT Filed: Mar. 25, 2013
(86) PCT No.: PCT/US2013/033658

§ 371 Date: Sep. 25, 2014

Related U.S. Application Data
(60) Provisional application No. 61/617,803, filed on Mar. 30, 2012.

(51) Int. Cl. C07C 227/16; C07C 229/20; C07C 227/18

(52) U.S. Cl. CPC C07C 227/16; C07C 227/18; C07C 229/20
USPC 560/172

(57) ABSTRACT

The instant invention describes a novel asymmetric synthesis of fluoroleucine alkyl esters which utilizes a phase transfer catalyst and a solid additive.

### BACKGROUND OF THE INVENTION

γ-Fluoroleucine-α-amino acids, alkyl esters and their derived peptides have been widely employed as potential pharmaceutical agents due to their broad biological properties, which include enzyme inhibitors, receptor antagonists and lipophilicity enhancing agents. While much development has focused on preparation of various fluorinated analogues of natural and non-proteinogenic amino acids, asymmetric synthesis of γ-fluoro-α-amino acids still remains a challenge. In this regard, stereoselective incorporations of the γ-F-containing side chain have been mostly executed by either chiral auxiliary-directed diastereoselective alkylation, chiral phase transfer-catalyzed alkylation of protected amino acid precursors or enzymatic hydrolysis of suitable precursors.

The instant invention describes a novel asymmetric synthesis of fluoroleucine alkyl esters which utilizes a phase transfer catalyst and a solid base in combination with a solid additive. Previously described phase transfer catalyzed alkylations of N-protected glycine derivatives utilized the glycine t-butyl ester derivative presumably to prevent the hydrolysis that can occur with other, less hindered ester moieties under phase transfer catalyzed reaction conditions. Previously described alkylations of N-protected glycine derivatives also utilized alkyl bromide under phase transfer catalyzed reaction conditions. Utilization of either the glycine t-butyl ester derivative or the alkyl bromide as raw materials for the synthesis of γ-fluoroleucine-α-amino acid is not viable for all purposes. In contrast, the present invention allows the use of readily accessible ethyl ester as the glycine equivalent and a commercially viable alkyl chloride as an alkylating reagent. Under these conditions, alkylation proceeds to completion with a catalyst loading as low as 0.1 mol % and good enantioselectivity.

### SUMMARY OF THE INVENTION

By this invention, there are provided processes for the preparation of compounds of structural formula I:

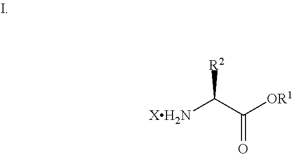

comprising the steps of:

a. alkylating an imine carboxylate of formula II with an alkylallyl halide, a phase transfer catalyst and a solid additive to form a substituted imine of formula III;

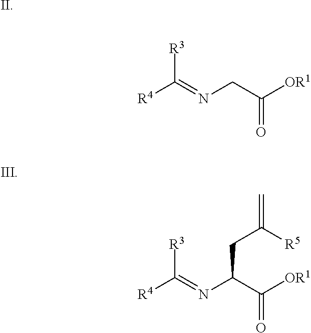

b. deprotecting the substituted imine of formula III with a protic acid to yield an amine of formula IV or a salt of formula V;

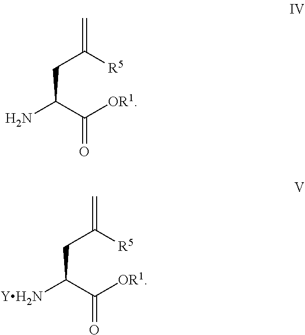

c. fluorinating the amine of formula IV or the salt of formula V to yield the compound of formula I.

wherein R1 is C₁₋₅ alkyl;
R² is C₁₋₅ fluoroalkyl;
R³ is aryl or heteroaryl;
R⁴ is aryl or heteroaryl;
R⁵ is C₁₋₃ alkyl;
X is H₂SO₄·½ pyridine;
Y is halide, sulfonate, phosphate or carboxylate.

### DETAILED DESCRIPTION OF THE INVENTION

By this invention, there are provided processes for the preparation of compounds of structural formula I:

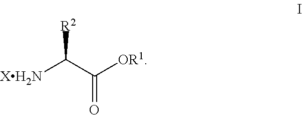

comprising the steps of:

a. alkylating an imine carboxylate of formula II with an alkylallyl halide, a phase transfer catalyst and a solid additive to form a substituted imine of formula III;

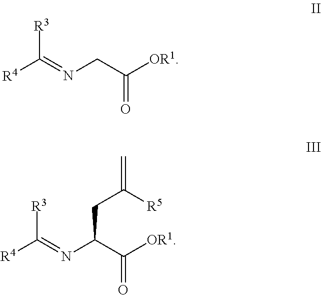

b. deprotecting the substituted imine of formula III with a protic acid to yield an amine of formula IV or a salt of formula V;

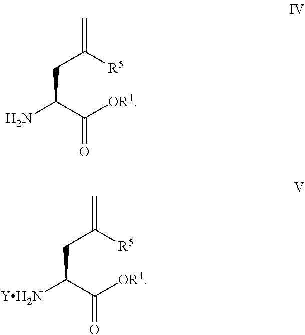

c. fluorinating the amine of formula IV or the salt of formula V to yield the compound of formula I,

wherein R¹ is C₁₋₅ alkyl;
R² is C₁₋₅ fluoroalkyl;
R³ is aryl or heteroaryl;
R⁴ is aryl or heteroaryl;
R⁵ is C₁₋₃ alkyl;
X is H₂SO₄½ pyridine;
Y is halide, sulfonate, phosphate or carboxylate.

In an embodiment of the invention, R¹ is ethyl.

In an embodiment of the invention, R³ is aryl. In a class of the invention, R³ is phenyl.

In an embodiment of the invention, R⁴ is aryl. In a class of the invention, R⁴ is phenyl.

In an embodiment of the invention, R⁵ is methyl.

In an embodiment of the invention, Y is halide. In another embodiment of the invention, Y is sulfonate. In a class of the invention, the sulfonate is naphthalenedisulfonate. In another embodiment of the invention, Y is phosphate. In another embodiment of the invention, Y is carboxylate.

In an embodiment of the invention, there are provided processes for the preparation of compounds of structural formula IA:

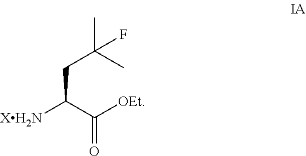

comprising the steps of:

a. alkylating an imine carboxylate of formula IIA with methallyl halide, a phase transfer catalyst and a solid additive to form a substituted imine of formula IIIA;

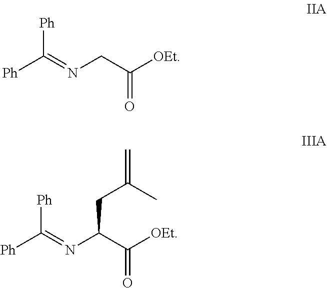

b. deprotecting the substituted imine of formula IIIA with a protic acid to yield an amine of formula IVA or a salt of formula VA;

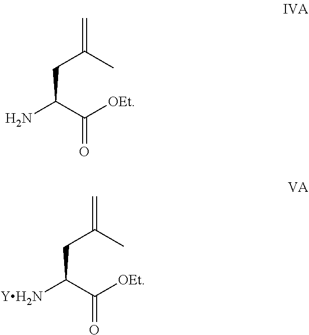

c. fluorinating the amine of formula IVA or the salt of formula VA to yield the compound of formula IA;

wherein X is H₂SO₄·½ pyridine;
Y is halide, sulfonate, phosphate or carboxylate.

An imine carboxylate of formula II is combined with an alkylallyl halide, a phase transfer catalyst and a solid additive in the presence of a solid base to form a substituted imine of formula III. This step utilizes a sub-stoichiometric amount of chiral phase transfer catalyst with the typical chemical yield of 90% or above. The efficiency of this asymmetric alkylation step is greatly enhanced by the use of a solid base of a particular particle size in combination with a solid additive of a particular particle size; the use of a solid additive helps the alkylation to proceed much more reproducibly at lower the catalyst loading compared to the reactions without one.

In one class of the invention, the solid base is sodium hydroxide, lithium hydroxide, potassium hydroxide, cesium hydroxide, rubidium hydroxide, calcium hydroxide, barium hydroxide, lithium tert-butoxide, sodium tert-butoxide, potassium tert-butoxide or mixtures thereof. In a subclass of the invention, the solid base is sodium hydroxide.

In one embodiment of the invention the particle size of the solid base is 20-50 um. In an embodiment of the invention, the phase transfer catalyst is an optically-active quaternary ammonium salt phase-transfer catalyst. In a class of the invention, the optically-active quaternary ammonium salt phase-transfer catalyst is described in United States Patent Application Publication US2009/0054679 to Nagase & Co, Ltd. Representative optically-active quaternary ammonium salt phase transfer catalysts include, but are not limited to, the following:

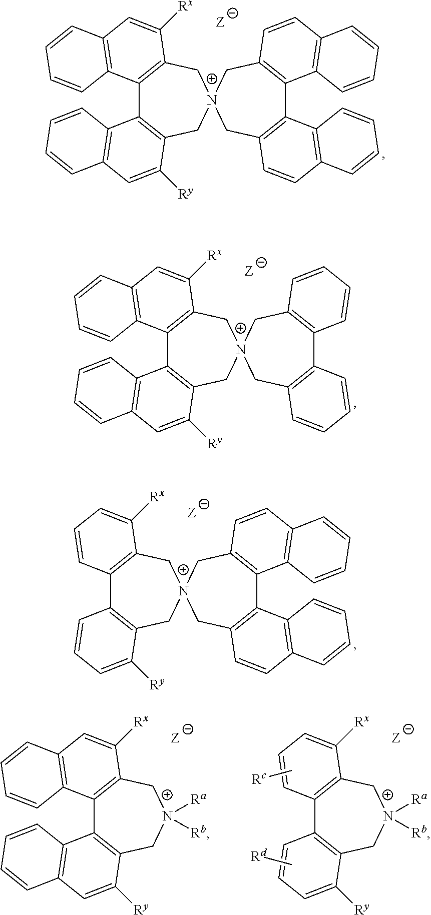

wherein Z is a halide anion; R^{x}, R^{y}, R^{a}, R^{b}, R^{c} and R^{d} are each independently selected from the group consisting of hydrogen, optionally substituted amine, cyano, nitro, carbamoyl, halo, C₁₋₆ alkyl (which is optionally substituted with halo), C₂₋₆ alkenyl (which is optionally substituted with halo), C₂₋₆ alkynyl (which is optionally substituted with halo), aryl (which is optionally substituted with halo, alkyl or haloalkyl), optionally substituted heteroaryl, optionally substituted heterocyclyl, and the like. In a subclass of the invention, the phase transfer catalyst is:

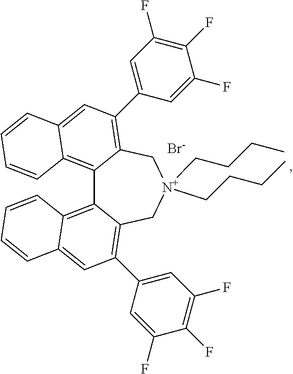 [SMILES: CCCC[N+]1(CCCC)Cc2c(-c3cc(F)c(F)c(F)c3)cc3ccccc3c2-c2c(c(-c3cc(F)c(F)c(F)c3)cc3ccccc23)C1 ; [Br-]]

which is available from Nagase Corporation (Japan).

In a class of the invention, the solid additive is aluminum oxide, sodium chloride, sodium bromide, magnesium sulfate, sodium fluoride, sodium iodide, sodium tetrafluoroborate, sodium hexafluorophosphate, sodium methanesulfonate, sodium benzenesulfonate, sodium trifluoromethanesulfonate, sodium carbonate, sodium phosphate, sodium sulfate or mixtures thereof. In a subclass of the invention, the solid additive is aluminum oxide. In another subclass of the invention, the solid additive is sodium chloride.

In one embodiment of the invention the particle size of the additive is 20-50 um. In one class of the invention, the combination described in step a. is performed at a temperature of about −70° C. to about 40° C. In a subclass of the invention, the temperature is −5° C. to 5° C.

The substituted imine of formula III is deprotected with protic acid to yield an amine of formula IV or a salt of formula V. In a class of the invention, the protic acid is 1,5-naphthalenedisulfonic acid, +p-toluenesulfonic acid (p-TSA), camphorsulfonic acid (CSA), methanesulfonic acid, hydrochloric acid, sulfuric acid, phosphoric acid or mixtures thereof. In a subclass of the invention, the protic acid is 1,5-naphthalenedisulfonic acid.

After deprotection, the amine of formula IV or salt of formula V is fluorinated. Many fluorinating agents can be used in the present invention. In one class of the invention, the fluorinating agent is Olah's reagent (HF in the form of Pyridine·9HF), HF-triethylamine, HF-Urea, HF-melamine or HF can also be used. In a subclass of the invention, the fluorinating agent is Olah's reagent. The fluorinated amine is then treated with sulfuric acid to yield a compound of formula I.

As used herein, “alkyl” is intended to include both branched and straight-chain saturated aliphatic hydrocarbon groups having one to ten carbon atoms unless otherwise specified. For example, C₁-C₁₀, as in “C₁-C₁₀ alkyl” is defined to include groups having 1, 2, 3, 4, 5, 6, 7, 8, 9 or 10 carbons in a linear, branched, or cyclic arrangement. For example, “C₁-C₁₀ alkyl” specifically includes methyl, ethyl, propyl, butyl, pentyl, hexyl, heptyl, octyl, nonyl, decyl, and so on.

“Alkoxy” or “alkyloxy” represents an alkyl group as defined above, unless otherwise indicated, wherein said alkyl group is attached through an oxygen bridge.

As used herein, the term “alkenyl” refers to a non-aromatic hydrocarbon radical, straight or branched, containing from 2 to 10 carbon atoms and at least 1 carbon to carbon double bond. Preferably 1 carbon to carbon double bond is present, and up to 4 non-aromatic carbon-carbon double bonds may be present. Thus, “C₂-C₆ alkenyl” means an alkenyl radical having from 2 to 6 carbon atoms. Alkenyl groups include ethenyl, propenyl, butenyl and cyclohexenyl. As described above with respect to alkyl, the straight, branched or cyclic portion of the alkenyl group may contain double bonds and may be substituted if a substituted alkenyl group is indicated.

The term “alkynyl” refers to a hydrocarbon radical straight or branched, containing from 2 to 10 carbon atoms, unless otherwise specified, containing at least 1 carbon to carbon triple bond. Up to 3 carbon-carbon triple bonds may be present. Thus, “C₂-C₆ alkynyl” means an alkynyl radical having from 2 to 6 carbon atoms. Alkynyl groups include ethynyl, propynyl and butynyl. As described above with respect to alkyl, the straight, branched or cyclic portion of the alkynyl group may contain triple bonds and may be substituted if a substituted alkynyl group is indicated.

As used herein, “aryl” is intended to mean any stable monocyclic or bicyclic carbon ring of up to 12 atoms in each ring, wherein at least one ring is aromatic. Examples of such aryl elements include phenyl, naphthyl, tetrahydronaphthyl, indanyl, biphenyl, phenanthryl, anthryl or acenaphthyl. In cases where the aryl substituent is bicyclic and one ring is non-aromatic, it is understood that attachment is via the aromatic ring.

The term “heteroaryl”, as used herein, represents a stable monocyclic, bicyclic or tricyclic ring of up to 10 atoms in each ring, wherein at least one ring is aromatic and contains from 1 to 4 heteroatoms selected from the group consisting of O, N and S. Heteroaryl groups within the scope of this definition include but are not limited to: benzoimidazolyl, benzofuranyl, benzofurazanyl, benzopyrazolyl, benzotriazolyl, benzothiophenyl, benzoxazolyl, carbazolyl, carbolinyl, cinnolinyl, furanyl, indolinyl, indolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, pyranyl, pyrazinyl, pyrazolyl, pyridazinyl, pyridopyridinyl, pyridyl, pyrimidinyl, pyrrolyl, quinazolinyl, quinolyl, quinoxalinyl, tetrazolyl, tetrazolopyridyl, thiadiazolyl, thiazolyl, thienyl, triazolyl, dihydrobenzoimidazolyl, dihydrobenzofuranyl, dihydrobenzothiophenyl, dihydrobenzoxazolyl, dihydroindolyl, dihydroquinolinyl, methylenedioxybenzene, benzothiazolyl, benzothienyl, quinolinyl, isoquinolinyl, oxazolyl, and tetra-hydroquinoline. In cases where the heteroaryl substituent is bicyclic and one ring is non-aromatic or contains no heteroatoms, it is understood that attachment is via the aromatic ring or via the heteroatom containing ring, respectively. If the heteroaryl contains nitrogen atoms, it is understood that the corresponding N-oxides thereof are also encompassed by this definition.

The term “heterocycle” or “heterocyclyl” as used herein is intended to mean a 5- to 10-membered nonaromatic ring, unless otherwise specified, containing from 1 to 4 heteroatoms selected from the group consisting of O, N, S, SO, or SO₂ and includes bicyclic groups. “Heterocyclyl” therefore includes, but is not limited to the following: piperazinyl, piperidinyl, pyrrolidinyl, morpholinyl, thiomorpholinyl, tetrahydropyranyl, dihydropiperidinyl, tetrahydrothiophenyl and the like. If the heterocycle contains a nitrogen, it is understood that the corresponding N-oxides thereof are also emcompassed by this definition.

As appreciated by those of skill in the art, “halo” or “halogen” as used herein is intended to include chloro, fluoro, bromo and iodo. The term “keto” means carbonyl (C═O). The term “alkoxy” as used herein means an alkyl portion, where alkyl is as defined above, connected to the remainder of the molecule via an oxygen atom. Examples of alkoxy include methoxy, ethoxy and the like.

The term “haloalkyl” means an alkyl radical as defined above, unless otherwise specified, that is substituted with one to five, preferably one to three halogen. Representative examples include, but are not limited to trifluoromethyl, dichloroethyl, and the like.

The present invention also includes compounds of structural formula IA:

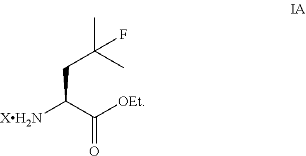

Wherein n X is H₂SO₄·½ pyridine.

In the examples below, various reagent symbols and abbreviations have the following meanings

PTC: phase transfer catalyst
1,5-NDSA: 1,5-naphthalenedisulfonic acid
IPA: isopropyl alcohol
HF: hydrogen fluoride
TFA: trifluoroacetic acid
MTBE: tert-butyl methyl ether
DBDMH: 1,3-dibromo-5,5-dimethylhydantoin

The following examples further illustrate the processes of the present invention, and details for the preparation of the compounds of the present invention. Those skilled in the art will readily understand that known variations of the conditions and processes of the following preparative procedures can be used to prepare these compounds. All temperatures are degrees Celsius unless otherwise noted.

### Example 1

### PTC Alkylation

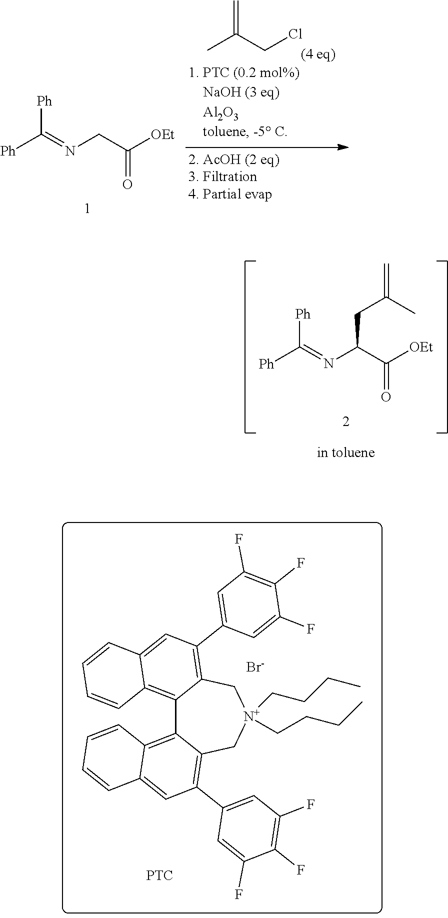

To a nitrogen-flushed 1L, 3-necked flask equipped with a mechanical stirrer was charged 1 (50 g, 183 mmol), PTC (0.274 g, 0.367 mmol), and degassed toluene (200 ml). The resulting mixture was cooled to −5° C. under nitrogen atmosphere. 3-chloro-2-methylpropene (73.2 ml, 733 mmol) was added followed by a mixture of pin-milled NaOH (ca. 40 um) and commercial aluminum oxide powder in one portion with the aid of degassed toluene (50 ml). The resulting light yellow mixture was stirred at −5° C. and degassed water (6.60 ml, 367 mmol) added over 2−11 hours. On complete reaction, NaOAc·3H₂O (1.0 g) was added. Acetic acid (20.46 ml, 357 mmol) was added drop-wise over 1 hour at −5° C. The resulting slurry was aged for 30 min and then filtered to remove solids. The cake was washed with toluene (250 mL). The filtrate was partially evaporated under vacuum to remove excess alkyl chloride. The final volume of the crude solution was ca. 200 ml.

### HPLC Assay=60.3 g, 88.6% ee

### Example 2

### Deprotection/Salt Formation

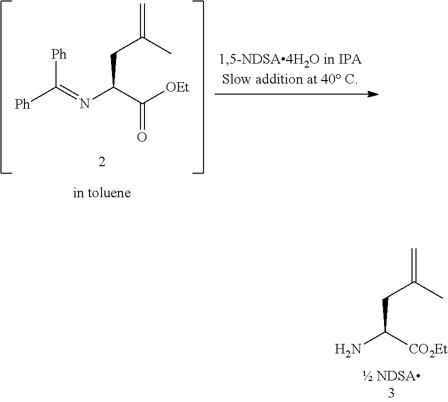

The solution of 2 (35 g, 109 mmol) in toluene (175 ml) was placed in a 500 ml, 3-necked flask equipped with a mechanical stirrer, and warmed to 40° C. To the solution was added a small amount of 3 as seed (20 mg), and then a solution of 1,5-NDSA (22.25 g, 59.9 mmol) in IPA (70.0 ml) was added slowly over 1 hour. The resulting off white slurry was aged at 40° C. for 10 min at which time HPLC showed that the deprotection was complete. Toluene (105 ml) was added slowly over 70 min, and the resulting slurry was aged at 40° C. for 40 min and cooled to room temperature. The solid was collected by filtration and the cake rinsed with 10% IPA in toluene (50 ml) followed by toluene (75 ml). The white solid thus obtained was dried under vacuum to afford 31.43 g of 3 (96.1 wt %, Y=92%, 89.1% ee).

### Example 3

### Fluorination/Salt Formation

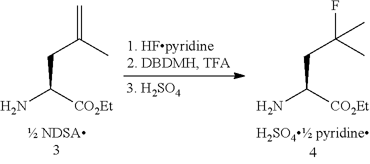

HF·pyridine (66.5 g, 2.33 mol) was placed in a 250 mL Teflon flask and cooled to 0-5° C. Compound 3 (36.9 g, 94.8 wt %, 116 mmol) was added over 15 minutes. After aging for 10 minutes, the cooling bath was removed and the reaction mixture was allowed to warm to room temperature. After aging for 2.5 hours, the mixture was cooled to below 5° C. and slowly added to a cold solution of NH₄OAc (179 g, 2.33 mol) in water (175 ml) over 30 minutes maintaining the internal temperature below 12° C. The pH was adjusted to 9 with KOH solution (45 wt %), the resulting slurry was filtered, and the solid was washed with MTBE (116 ml×3). The layers were separated, and the organic layer was washed with brine. The solution was assayed with HPLC to contain 15.9 g of product (89.7 mmol, 77% yield).

This crude solution was cooled to 5° C. with ice/water bath, water (15.9 ml), 1,3-dibromo-5,5-dimethylhydantoin (DBDMH, 2.57 g, 9 mmol) and TFA (13.8 ml, 179 mmol) were added. The reaction mixture was then aged at room temperature for 2.5 hours, after which it was acidified with 1N HCl (40 ml). The organic layer was separated and extracted with 1N HCl (40 ml). MTBE (160 ml) was added to the combined aqueous layers and the mixture cooled in an ice/water bath. NH₄OH was added to adjust the pH to 9. The aqueous layer was separated and extracted with fresh MTBE (60 ml×2). The combined organic layer was washed with brine and passed through a pad of MgSO₄. The filtrate was then concentrated under vacuum to a total volume of ca. 145 ml and acetonitrile (40 ml) added. This solution was cooled to ca. 15° C. and sulfuric acid (3.8 ml, 71.8 mmol) added. The resulting thick slurry was aged at room temperature overnight. The solid was collected by filtration, washed with 3:1 MTBE: acetonitrile (120 ml), and dried under vacuum to afford 4 as off-white solid (17.52 g, 55.6 mmol, 62% yield, 97.5% ee).

¹H NMR (400 MHz, DMSO) δ=8.58 (dt, J=4.2Hz, 1.7 Hz, 1.5 H), 8.49 (bs), 7.80 (tt, J=7.6 Hz, 1.7 Hz, 0.58 H), 7.41-7.38 (m, 1.1 H), 4.21 (q, J=7.2 Hz, 2 H), 4.17 (t, J=6.7 Hz, 1 H), 2.21 (ddd, J=23.1 Hz, 15.3 Hz, 6.6 Hz, 1 H), 2.08 (ddd, J=21.9 Hz, 15.1 Hz, 6.7 Hz, 1 H) 1.41 (d, J=21.6 Hz, 6 H), 1.24 (t, 7.2 Hz, 3 H)

What is claimed is:

1. A process for preparing a compound of formula I: 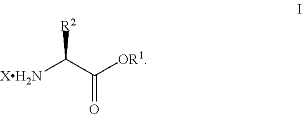
comprising the steps of:
a. alkylating an imine carboxylate of formula II with an alkylallyl halide, a phase transfer catalyst and a solid additive to form a substituted imine of formula III; 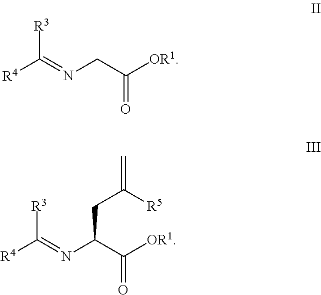
b. deprotecting the substituted imine of formula III with a protic acid to yield an amine of formula IV or a salt of formula V; 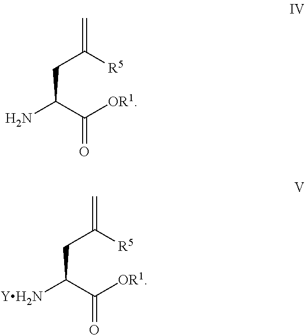
c. fluorinating the amine of formula IV or the salt of formula V to yield the compound of formula I,
wherein R¹ is C₁₋₅ alkyl;
R² is C₁₋₅ fluoroalkyl;
R³ is aryl or heteroaryl;
R⁴ is aryl or heteroaryl;
R₅ is C₁₋₃ alkyl;
X is H₂SO₄·½ pyridine;
Y is halide, sulfonate, phosphate or carboxylate.

2. The process of claim 1 wherein R¹ is ethyl; R³ is phenyl; R⁴ is phenyl; R⁵ is methyl.

3. The process of claim 1 wherein step a. is performed in the presence of a solid base.

4. The process of claim 3 wherein the solid base is selected from the group consisting of sodium hydroxide, lithium hydroxide, potassium hydroxide, cesium hydroxide, rubidium hydroxide, calcium hydroxide, barium hydroxide, lithium tert-butoxide, sodium tert-butoxide, potassium tert-butoxide and mixtures thereof

5. The process of claim 4 wherein the solid base is sodium hydroxide.

6. The process of claim 1 wherein the phase transfer catalyst is

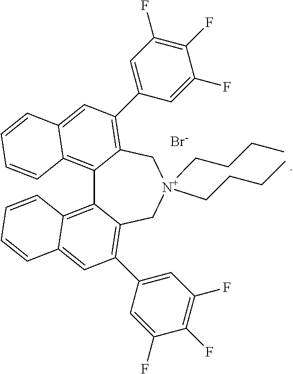 [SMILES: CCCC[N+]1(CCCC)Cc2c(-c3cc(F)c(F)c(F)c3)cc3ccccc3c2-c2c(c(-c3cc(F)c(F)c(F)c3)cc3ccccc23)C1 ; [Br-]]

7. The process of claim 1 wherein the solid additive is selected from the group consisting of aluminum oxide, sodium chloride, sodium bromide, magnesium sulfate, sodium fluoride, sodium iodide, sodium tetrafluoroborate, sodium hexafluorophosphate, sodium methanesulfonate, sodium benzenesulfonate, sodium trifluoromethanesulfonate, sodium carbonate, sodium phosphate, sodium sulfate and mixtures thereof

8. The process of claim 7 wherein the solid additive is aluminum oxide.

9. The process of claim 1 wherein step c. is performed with a fluorinating agent selected from Olah's reagent HF, HF-Urea, HF-melamine or a mixture thereof

10. The process of claim 9 wherein the fluorinating agent is Olah's reagent.

11. A compound of formula I 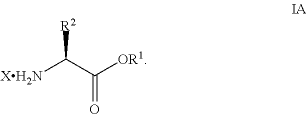
wherein X is H₂SO₄·½ pyridine.
